(19) United States
(12) Patent Application Publication
(10) Pub. No.: US 2011/0238202 A1
(43) Pub. Date: Sep. 29, 2011

(54) EQUIPMENT FOR TRANSFERRING ITEMS BY SUCTION, AND DISINFECTING METHOD

(75) Inventor: Olivier Somville, Cholet (FR)

(73) Assignee: BIOWIND GROUP, Le Chesnay (FR)

(21) Appl. No.: 13/133,607

(22) PCT Filed: Dec. 7, 2009
(86) PCT No.: PCT/FR09/52429

§ 371 Date: Jun. 8, 2011

(30) Foreign Application Priority Data
Dec. 8, 2008 (FR) 0858355

(51) Int. Cl. G06F 19/00; A61L 2/18

(52) U.S. Cl. 700/117; 422/28

(57) ABSTRACT

Equipment for transferring items by suction, having a control unit, a movable plate carrying a set of suction cups, drive means, and pneumatic aspiration means, the movable plate being moved by the drive means alternately between loading position and an unloading position. The equipment is further provided with spraying means designed and controlled by the control unit to spray a disinfectant product onto the suction cups when the movable plate has left its unloading position and before it has reached its loading position.

The invention relates in general to automatic packaging techniques, particularly in the area of food products.

More specifically, the invention relates, according to a first feature, to equipment for transferring items that are gripped by suction cups, said equipment having a control unit, a movable plate, drive means designed to move, in operation, the movable plate alternately between a loading position and an unloading position, a plurality of suction cups carried on the movable plate, and pneumatic suction means designed to selectively apply a negative pressure to the suction cups and including a negative pressure source such as a vacuum pump and pneumatic connecting elements, the control unit being designed to control the pneumatic suction means synchronously with the operation of the drive means and to reduce or interrupt application of the negative pressure to the suction cups in the unloading position of the movable plate.

Equipment of this type are well known in the prior art, and are applied in particular to packaging or sorting natural food products such as eggs or food products obtained by processing such as bakery items.

While, of course, these equipment require very strict hygiene conditions designed to prevent the development of pathogenic germs and molds, the use of the vacuum to grip the items easily leads to the entrainment and trapping of food particles favoring such development.

With present disinfection techniques, this difficulty is overcome by periodically gassing the area in which the transfer equipment is located using a disinfectant gas.

Other than the fact that this procedure is somewhat cumbersome and expensive, it appears that it is not totally effective.

In this context, the specific goal of the invention is to a propose a equipment for transferring items that are gripped by suction cups, designed to offer an even higher standard of hygiene.

For this purpose, the equipment according to the invention, otherwise conforming to the generic definition provided in the preamble hereinabove, is essentially characterized by additionally including spraying means including a spraying unit positioned and designed to spray a disinfectant product onto the suction cups of the movable plate in response to a spraying command, and by the control unit being designed to produce said spraying command at an intermediate position assumed by the movable plate in its return movement from its unloading position to its loading position.

The pneumatic connecting elements include for example at least one solenoid valve controlled by the control unit and selectively connecting the suction cups to the negative pressure source or to atmosphere.

Preferably, the spraying means additionally include a reservoir containing a disinfectant liquid constituting the disinfectant product, and a propulsion device controlled by the control unit to selectively spray the liquid through at least one nozzle in the spraying unit.

In one possible embodiment, the propulsion element is an electric pump functionally disposed between the reservoir and each nozzle.

In another possible embodiment, the propulsion element is a compressed air source and each nozzle of the spraying unit is a venturi nozzle having a compressed air inlet and a disinfectant liquid inlet.

In the latter case, the spraying means advantageously has a solenoid valve functionally disposed between the compressed air source and the compressed air inlet of each nozzle, possibly as well as a solenoid valve functionally disposed between the reservoir and the disinfectant liquid inlet of each nozzle.

The equipment of the invention may also include an ozone generator designed to dissolve ozone by bubbling it into the liquid contained in the reservoir, in which case the disinfectant product can be essentially ozone-laden water.

The invention also relates to a method for disinfecting a equipment for transferring items provided with pneumatic suction means and suction cups each of which is connected to the suction means and designed to carry an item during an active transfer phase, this method being characterized by including an operation consisting of spraying a disinfectant product onto the suction cups outside the active transfer phase and during a phase in which the suction means are operational.

In one advantageous embodiment of this method, the disinfectant product is liquid.

Other characteristics and advantages of the invention will emerge clearly from the description hereinbelow, provided indicatively and not exhaustively, with reference to the attached drawings wherein:

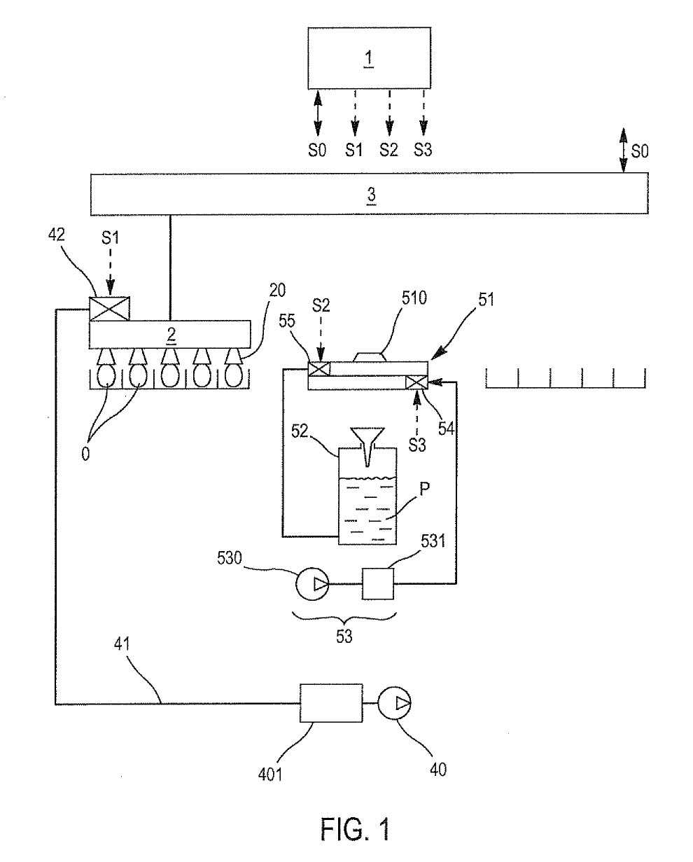

FIG. 1 is a schematic view of a transfer equipment according to the invention wherein the movable plate is shown in its loading position;

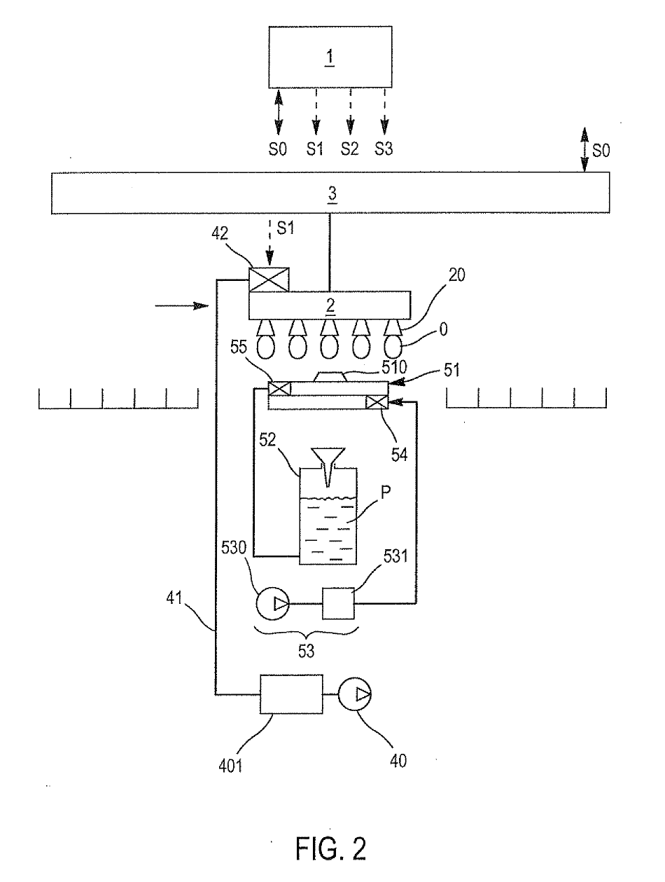

FIG. 2 is a schematic view of the transfer equipment shown in FIG. 1, wherein the movable plate is shown in a position intermediate between its loading position and its unloading position;

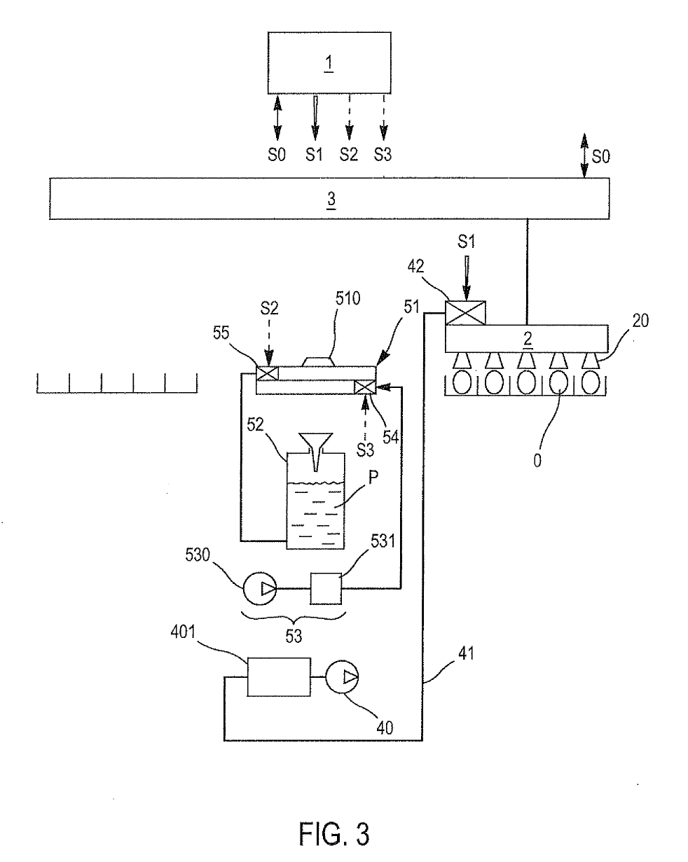

FIG. 3 is a schematic view of the transfer equipment shown in FIG. 1, wherein the movable plate is shown in its unloading position;

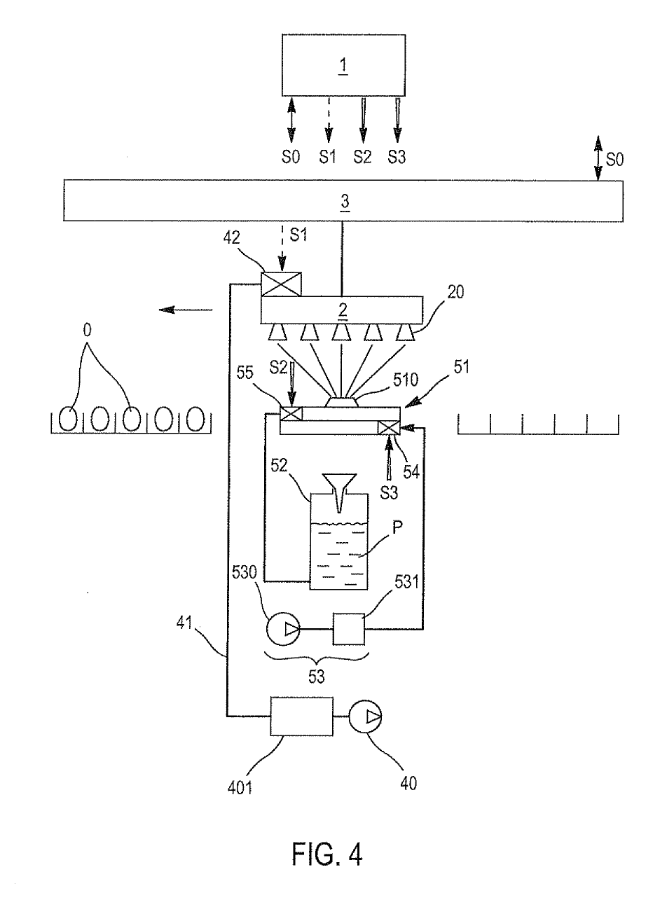

FIG. 4 is a schematic view of the transfer equipment shown in FIG. 1, wherein the movable plate is shown in a position intermediate between its unloading position and its loading position;

FIG. 5 is a schematic view of a venturi nozzle usable in the transfer equipment shown in FIG. 1;

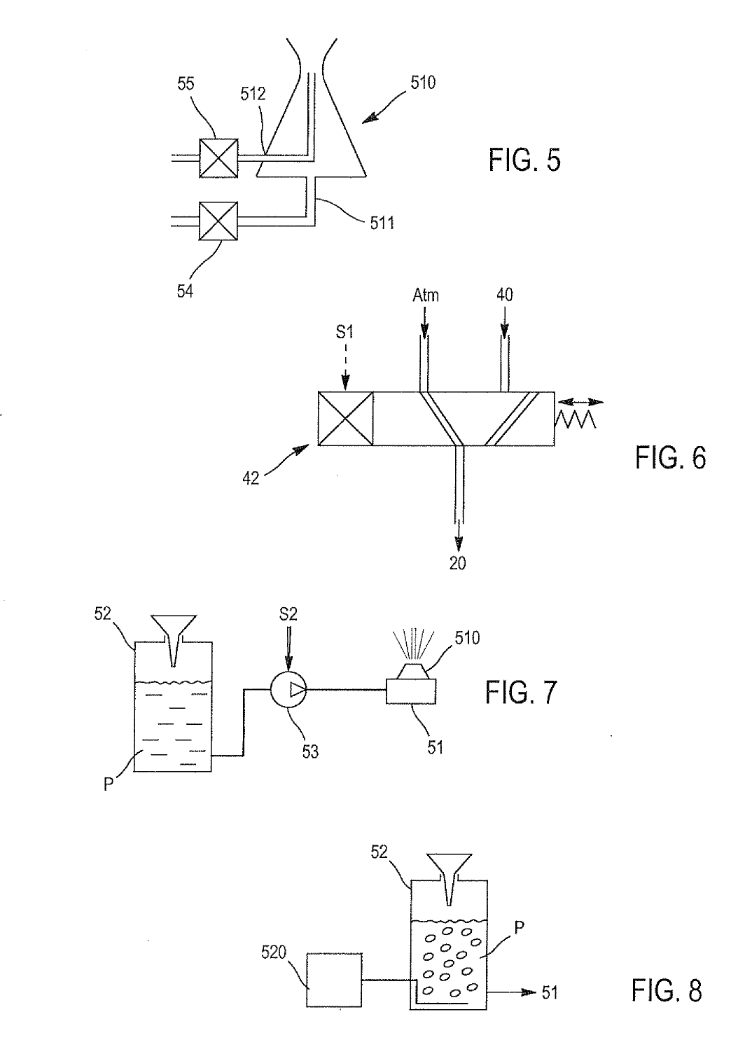

FIG. 6 is a schematic view of a solenoid valve belonging to the pneumatic linking elements of the equipment, said solenoid valve being shown in the configuration it assumes then the movable plate is in its unloading position;

FIG. 7 is a schematic view of an alternative embodiment of the spraying means usable in equipment according to the invention; and

FIG. 8 is a schematic view of a disinfectant liquid production unit usable in equipment according to the invention.

As stated above, the invention relates to a equipment for transferring items by suction, wherein the figures show the transfer of eggs 0.

Such a equipment includes in particular a control unit 1, a movable plate 2 having a set of suction cups 20, drive means 3, and pneumatic suction means such as 40 to 42.

In operation, the drive means 3 that move the movable plate 2 alternately between a loading position visible in FIG. 1 and an unloading position shown in FIG. 3, passing in both the forward and return directions through intermediate positions visible in FIGS. 2 and 4, respectively.

The pneumatic suction means, which are designed to apply a negative pressure to the suction cups 20 when the movable plate 2 is in its unloading position, have a negative pressure source 40, which is a vacuum pump for example, and pneumatic connecting elements such as pipes 41 and one or more solenoid valves 42.

A vacuum reserve 401, which may also serve as a filter and trap, may be provided between the vacuum pump 40 and the suction cups 20.

The control unit 1, which is for example a programmable robot, is designed to control the pneumatic suction means 40 to 42 synchronously with the operation of the drive means 3, particularly to reduce or interrupt the application of the negative pressure to the suction cups 20 when the movable plate 2 is in its unloading position.

For this purpose, the control unit 1 and the drive means 3 exchange for example data S0 that enable at least control unit 1 to recognize the momentary position of the movable plate 2.

For its part, the solenoid valve 42 (FIGS. 1 to 4 and 6) is designed for example to connect the suction cups 20 to the vacuum pump 40 by default, and connect these suction cups to atmosphere Atm in response to a control signal S1 emitted by the control unit 1 when the movable plate 2 reaches its unloading position.

The equipment according to the invention is also provided with spraying means 51 to 55 designed to spray a disinfectant P onto the suction cups 20 when the movable plate 2 has left its unloading position and before it reaches its loading position, namely when this plate is in an intermediate return position as shown in FIG. 4.

For this purpose, these spraying means have a spraying unit 51 positioned in the return path of the movable plate 2 and designed to spray the disinfectant product P onto the suction cups 20 of the movable plate 2 in response to a spraying command produced by the control unit 1.

For its part, the control unit 1, which is informed of the position of movable plate 2 by the data SO coming from drive means 3, is designed to produce the spraying command when the movable plate 2 is opposite the spraying unit 51, as shown in FIG. 4.

As shown in particular in FIGS. 1 to 4, the spraying means preferably have a reservoir 52 containing a disinfectant liquid P constituting the disinfectant product.

This liquid P is sprayed on command through one or more nozzles 510 of the spraying unit 51 by means of a propulsion element 53 under the control of the control unit 1.

In the embodiment specifically illustrated in FIG. 7, the propulsion element 53 is comprised of an electric pump functionally disposed between the reservoir 52 and the nozzle or nozzles 510, said pump being started on reception of a spraying command S2 delivered by the control unit 1.

In the embodiment shown in FIGS. 1 to 5, the propulsion element 53 is a compressed air source and the nozzle or each of the nozzles 510 of the spraying unit 51 is a venturi nozzle having a compressed air inlet 511 and a disinfectant liquid inlet 512 (FIG. 5).

The compressed air source is comprised for example of a pump 530 provided with a pressure reserve 531.

The function performed by the pump 530 can be provided by the vacuum pump 401 provided this pump is regulated, said regulation enabling the amount of waste air discharged by the vacuum pump to be reduced.

The flowrate of the compressed air source 53 can be controlled by a solenoid valve 54 functionally disposed between the compressed air reserve 531 and the compressed air inlet 511 of each nozzle 510, said solenoid valve being controlled by a control signal S3 emitted by the control unit 1.

Where applicable, the spraying means can also have a solenoid valve 5, functionally disposed between the reservoir 52 and the disinfectant liquid inlet 512 of each nozzle 510, and controlled by a control signal S2 emitted by the control unit 1.

As will readily be understood by the individual skilled in art upon reading the foregoing, the spraying command delivered by the control unit 1 in the embodiment of FIGS. 1 to 5 can thus be comprised of control signals S2 and S3, while it is comprised only of a single signal S2 in the embodiment described with reference to FIG. 7.

As shown in FIG. 8, the equipment of the invention can also comprise an ozone generator 520 connected to the reservoir 52 and designed to produce ozone which is dissolved by bubbling it into the liquid P contained in the reservoir 52.

Such an arrangement, which exploits the remarkable bactericidal efficacy of ozone combined with its short lifetime, enables a cheap and effective disinfectant with no long-term harmful effect on the environment to be obtained using water for example as the base liquid.

Whatever the embodiment used to implement the invention, the latter has the decisive advantage of allowing not only the suction cups but also all the pneumatic suction means to be effectively disinfected, including pump 40 and vacuum reserve 401 where it exists.

1. Equipment for transferring items gripped by suction cups, said equipment comprising:
a control unit, a movable plate, drive means designed to move, in operation, the movable plate alternately between a loading position and an unloading position, a plurality of suction cups carried on the movable plate, pneumatic aspiration means designed to selectively apply a negative pressure to the suction cups and including a negative pressure source, the control unit being designed to control the pneumatic aspiration means synchronously with the operation of drive means to reduce or interrupt the application of negative pressure to the suction cups in the unloading position of the movable plate, and spraying means including a spraying unit positioned and designed to spray a disinfectant product onto the suction cups of the movable plate in response to a spraying command, wherein the control unit is designed to produce said spraying command at an intermediate position assumed by the movable plate in its return movement from its unloading position to its loading position.

2. Equipment according to claim 1, wherein the pneumatic connecting means include at least one solenoid valve controlled by the control unit selectively connecting the suction cups to the negative pressure source or to atmosphere.

3. Equipment according to claim 1, wherein the spraying means additionally include a reservoir containing a disinfectant liquid constituting the disinfectant product, and a propulsion element controlled by the control unit to selectively spray the liquid through at least one nozzle of the spraying unit.

4. Equipment according to claim 3, wherein the propulsion element is an electric pump functionally disposed between the reservoir and each nozzle.

5. Equipment according to claim 3, wherein the drive element is a compressed air source and each nozzle of the spraying unit is a venturi nozzle having a compressed air inlet and a disinfectant liquid inlet.

6. Equipment according to claim 5, wherein the spraying means additionally include a solenoid valve functionally disposed between the compressed air source and the compressed air inlet of each nozzle.

7. Equipment according to claim 5, wherein the spraying means additionally include a solenoid valve functionally disposed between the reservoir and the disinfectant liquid inlet of each nozzle.

8. Equipment according to claim 3, further comprising an ozone generator designed to dissolve ozone by bubbling it into the liquid contained in the reservoir.

9. Method for disinfecting a equipment for transferring items provided with pneumatic suction means and suction cups each of which is connected to the suction means and designed to carry an item during an active transfer phase, this method being characterized by including an operation comprising spraying a disinfectant product onto the suction cups outside the active transfer phase and during a phase in which the suction means are operational.

10. Disinfection method according to claim 9, wherein said disinfectant product is liquid.
